(19) United States
(12) Patent Application Publication
(10) Pub. No.: US 2007/0162075 A1
(43) Pub. Date: Jul. 12, 2007

(54) Method and apparatus for broadcasting audible information prompts from an external defibrillator

(75) Inventor: Richard Alan O'Hara, Bellevue, WA (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 10/598,091

(22) PCT Filed: Feb. 1, 2005
(86) PCT No.: PCT/IB05/50428

§ 371 Date: Aug. 17, 2006

Related U.S. Application Data
(60) Provisional application No. 60/545,820, filed on Feb. 19, 2004.

(51) Int. Cl. A61N 1/00

(52) U.S. Cl. 607/5

(57) ABSTRACT

A method and apparatus provides instructions to a user for operating an external defibrillator device having a set of electrodes coupleable to a patient. The method begins by transmitting over a wireless protocol a voice prompt instructing the user to attach the set of electrodes to the patient. The method continues by transmitting over the wireless protocol at least one additional voice prompt instructing the user to administer defibrillator therapy.

The present invention relates in general to defibrillators, and more particularly to automatic or semi-automatic external defibrillators (“AED”).

Sudden cardiac death is the leading cause of death in the United States, with one person dying every two minutes. Most sudden cardiac death is caused by ventricular fibrillation (“VF”), in which the heart's muscle fibers contract without coordination, thereby interrupting normal blood flow to the body. The only known effective treatment for VF is electrical defibrillation, in which an electrical pulse is applied to the patient's heart. The electrical pulse must be delivered within a short time after onset of VF in order for the patient to have any reasonable chance of survival. Electrical defibrillation may also be used to treat shockable ventricular tachycardia (“VT”). Accordingly, defibrillation is the appropriate therapy for any shockable rhythm, i.e., VF or shockable VT.

One way of providing electrical defibrillation uses an external defibrillator. External defibrillators send electrical pulses to the patient's heart through electrodes applied to the patient's torso. External defibrillators are typically located and used in hospital emergency rooms, operating rooms, and emergency medical vehicles. Of the wide variety of external defibrillators currently available, automatic and semi-automatic external defibrillators (referred to collectively as “AEDs”) are becoming increasingly popular because they can be used by relatively inexperienced personnel. Such AEDs are also especially lightweight, compact, and portable. AEDs are described, for example, in U.S. Pat. No. 5,607,454 to Cameron et al. entitled “Electrotherapy Method and Apparatus,” PCT Publication No. WO 94/27674 entitled “Defibrillator with Self-Test Features,” and U.S. Published Patent Application No. 2002/0156503 entitled “Method and Apparatus for Providing On-Screen Incident Review in an AED.”

AEDs provide a number of advantages, including the availability of external defibrillation at locations where external defibrillation is not regularly expected, and is likely to be performed quite infrequently, such as in residences, public buildings, businesses, personal vehicles, public transportation vehicles, etc. This is important because the chances of surviving a cardiac arrest decrease rapidly over the time following the arrest. Quick response to a cardiac arrest by performing CPR and by administering a defibrillating shock is therefore of critical importance.

AEDs differ from manual defibrillators in that AEDs can automatically analyze the electrocardiogram (ECG) rhythm to determine if defibrillation is necessary. In nearly all AED designs, the first responder is prompted to press a shock button to deliver the defibrillation shock to the patient. Paramedic defibrillators often combine the AED and manual functions into one unit to allow for use by personnel with differing levels of training.

AEDs are designed to be used primarily by first responders who may not be trained in proper advanced cardiac life support (ACLS) techniques. In the pre-hospital setting, these first responders may include emergency medical technicians trained in defibrillation (EMT-Ds), police officers, flight attendants, security personnel, occupational health nurses, and firefighters. AEDs can also be used in areas of the hospital where personnel trained in ACLS are not readily available.

Even if the first responder does have some basic training in device operation and cardiopulmonary resuscitation (CPR), he or she may forget this basic training during the stress of reacting to a heart attack. With wider deployment of AEDs in homes and public venues, the minimally trained or even untrained use of defibrillation devices will increase. Accordingly, AEDs should be able to successfully direct precise instructions to a first responder with minimal or no training through a cardiorespiratory event, i.e., CPR as well as AED device operation.

AEDs are currently available that provide the first responder with instructions for deploying the defilbrillator and administering CPR to a patient. For example, the AED disclosed in U.S. Pat. No. 6,334,070 provides visual and aural instructions to the first responder via a display and speakers that are embedded in the AED housing. Since AEDs are generally portable devices, the size of the speakers is limited. Consequently, since small speakers cannot be driven very hard, the volume and fidelity of the speakers are also limited. This can present serious problems when the AED is used in a noisy environment, frustrating the first responder's ability to adequately comprehend and follow the aural prompts. For example, AEDs are commonly deployed and used on commercial aircraft, where high levels of ambient noise drown out the aural instructions that are issued from the AED during a rescue. Another problem with aural prompts is that they can interfere with the recording of background conversations occurring during the rescue process. Such recordings are often required by fire departments or other public agencies during a rescue operation, in order to conduct post-rescue analysis. Frequent aural prompts issued by the AED can disrupt or drown out the running commentary.

In accordance with the present invention, a method and apparatus provides instructions to a user for operating an external defibrillator device having a set of electrodes couplable to a patient. The method begins by transmitting over a wireless protocol a voice prompt instructing the user to attach the set of electrodes to the patient. The method continues by transmitting over the wireless protocol at least one additional voice prompt instructing the user to administer defibrillator therapy.

In accordance with one aspect of the invention, a voice prompt is transmitted instructing the user to administer CPR therapy.

In accordance with another aspect of the invention, a voice prompt is transmitted instructing the user that a patient assessment sequence is to begin.

In accordance with another aspect of the invention, the external defibrillator is a fully automatic external defibrillator.

In accordance with another aspect of the invention, the external defibrillator is a semi-automatic external defibrillator.

In accordance with another aspect of the invention, the wireless protocol is selected from the group consisting of Bluetooth, IEEE 802.11, IEEE 802.15, IEEE802.16, Near Field Communication—Interface and Protocol (“NFCIP-1”), and HomeRF.

In accordance with another aspect of the invention, the voice prompts are transmitted to a receiver embedded in a portable device.

In accordance with another aspect of the invention, the portable device is selected from the group consisting of a headphone, wireless telephone and a PDA.

In accordance with another aspect of the invention, an electrotherapy device is provided that includes a controller, an energy source, at least one electrode for providing electrotherapy to a patient, and an energy delivery system operable by the controller to deliver an electrical shock from the energy source to the at least one electrode. The electrotherapy device also includes a voice circuit for generating audio prompts initiated by the controller and a wireless transmitter coupled to the voice circuit for transmitting the audio prompts over a wireless communication protocol.

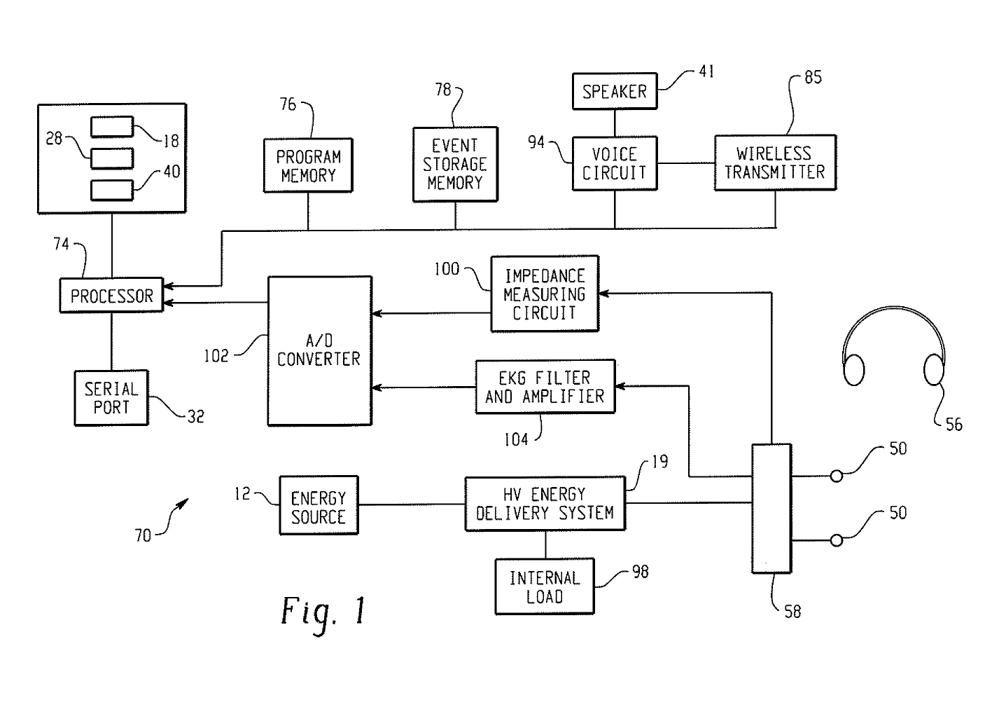

FIG. 1 is a block diagram of one embodiment of an electrotherapy device constructed in accordance with the present invention.

The following discussion is presented to enable a person skilled in the art to make and use the invention. Various modifications to the preferred embodiment will be readily apparent to those skilled in the art, and the generic principles defined herein may be applied to other embodiments and applications without departing from the spirit and scope of the present invention as defined by the appended claims. Thus, the present invention is not intended to be limited to the embodiment shown, but is to be accorded the widest scope consistent with the principles and features disclosed herein.

The present invention provides an automatic or semiautomatic external defibrillator that overcomes the above-mentioned problems by transmitting the aural prompts required by the first responder or other user over a wireless transmitter to a wireless receiver located in a pair of headphones that can be worn by the user. In this way the user can be assured that he or she will clearly receive the aural information with a minimum of disruption from other ambient sources. The defibrillator may or may not also include a speaker built into the unit to broadcast the prompts. If a speaker is included, in some embodiments of the invention the speaker may be muted manually, or in some cases automatically when the user initiates a wireless transmission mode.

While the transmission of responder prompt information over a wireless connection to a headphone worn by the responder in accordance with the present invention is applicable to any automatic or semiautomatic external defibrillator, for purposes of illustration only one particular automatic external defibrillator will be described in connection with FIG. 1. Similarly, the particular voice prompts that are described are for illustrative purposes only and should not be construed as a limitation on the invention.

FIG. 1 is a block diagram of the electrical system of an automatic or semi-automatic external defibrillator 70. The overall operation of defibrillator 70 is controlled by a digital microprocessor-based control system that includes a processor 74 interfaced to program memory 76 and event memory 78. The operating program executed by processor 74 is stored in program memory 76. Of course, the operating program may be embodied in hardware, software, firmware, or any combination thereof. Electrical power is provided by an energy source 12 such as a rechargeable battery cartridge. A high voltage energy delivery system 19 is connected to receive power from the energy source 12.

High voltage energy delivery system 19 is also connected to and controlled by processor 74. Such energy delivery systems 19 are generally known, and disclosed for example, in U.S. Pat. No. 5,405,361. In response to charge control signals provided by the processor 74, high voltage energy delivery system 19 is operated in a charge mode during which one set of semiconductor switches (not separately shown) cause a plurality of capacitors (also not shown), to be charged by energy source 12. Once charged, and in response to discharge control signals provided by processor 74, high voltage energy delivery system 19 is operated in a discharge mode during which the capacitors are discharged in series by another set of semiconductor switches (not separately shown) to produce the high voltage defibrillation pulses. The defibrillation pulses are applied to the patient through electrodes 50, which are connected to the high voltage energy delivery system 19 by connector 58. Under certain circumstances described below, processor 74 causes high voltage generation circuit 86 to be discharged through an internal resistive load 98 rather than electrodes 50.

An impedance measuring circuit 100 is connected to the electrodes 50 and is interfaced to processor 74 through analog-to-digital (A/D) converter 102. The impedance measuring circuit 100 applies a signal to electrodes 50. The magnitude of the signal received back from the electrodes 50 is monitored by impedance measuring circuit 100. An impedance signal representative of the impedance present across connector 58 is then generated by circuit 100 as a function of the ratio of the magnitudes of the applied and received signals (i.e., the attenuation of the applied signal). For example, if the electrodes 50 are operational, a relatively low resistance (e.g., less than about ten ohms) should be present across the connector 58. If, for example, the conductive adhesive on the electrodes 50 is dried out, the electrodes 50 are not properly connected to connector 58, or the electrodes 50 are not properly positioned on the patient, a relatively high resistance (e.g, greater than about one hundred ohms) will be present. The impedance signal representative of the impedance measured by circuit 100 is digitized by A/D converter 102 and provided to processor 74.

Defibrillator 70 also includes electrocardiogram (EKG) filter and amplifier circuit 104 which is connected between electrodes 50 and AID converter 102. The EKG or cardiac rhythm of the patient is processed by filter and amplifier circuit 104 in a conventional manner, and digitized by A/D converter 102 before being coupled to processor 74.

Data communication port 32 is coupled to processor 74 for two-way serial data transfer using, for example, an RS-232 protocol. A diagnostic display located on the AED housing includes features such as a rescue switch 40, rescue switch light 28, and resume switch 18. A voice circuit 94 is connected to a wireless transmitter 85. In response to voice prompt control signals from processor 74, voice circuit 94 and wireless transmitter 85 generate voice prompts over a wireless carrier (e.g., an IR or RF carrier) to a receiver embedded in a headphone 56 that can be worn by the responder. In addition, in some embodiments of the invention the voice circuit 94 may also be connected to a speaker 34. The speaker 34 may generate audible voice prompts that can be heard by responders not wearing the wireless headphone.

In some embodiments of the invention the receiver may be embedded in a portable device other than a headphone. For example, the receiver may be embedded in a wireless telephone, personal digital assistant (PDA), or the like.

Wireless transmitter 85 and the receiver (not shown) located in the headphone 56 comply with an established communication standard. Of course, a wide variety of wireless protocols can be implemented, which may operate at a variety of different communication frequencies. In some embodiments of the invention the wireless transmitter 85 may send the voice prompts over a broadcastable wireless protocol, such as Bluetooth, IEEE 802.11, IEEE 802.15, IEEE802.16, Near Field Communication—Interface and Protocol (“NFCIP-1”), and HomeRF, for example. The Bluetooth protocol may be preferable in some embodiments of the invention because it typically consumes less power than other technologies.

In use, a rescue mode of operation is initiated when an operator removes the defibrillator protective cover to access the electrodes 50 and the headphones 56, which may be located in the same or different compartments of the defibrillator. Processor 74 then begins its rescue mode operation by initiating the generation of a voice prompt over the wireless transmitter 85 “To attempt a rescue, disconnect charger,” if a charger is connected to energy source 12 when the cover is removed. Prior to the generation of the voice prompt, processor 74 may go though a self-test procedure checking such items as the charge state of the batteries, the interconnection and operability of electrodes 50, the state of event storage memory 78, and the functionality of A/D converter 102.

The processor 74 initiates the generation of a “Place electrodes” voice prompt over the wireless transmitter 85. In response to this voice prompt, the operator should place the electrodes on the patient's chest. While this action is being performed, processor 74 monitors the impedance signals received through A/D converter 102 to determine whether the impedance across the electrodes indicates that they have been properly positioned on the patient. If the correct impedance is not measured, processor 74 initiates the generation of a “Check electrodes” voice prompt.

After detecting an impedance indicating the proper placement of electrodes 50, and without further action by the operator (i.e., automatically), processor 74 begins a first analyze sequence by initiating the generation of a “Do not touch patient. Analyzing rhythm” voice prompt, and analyzing the patient's cardiac rhythm. In one embodiment, processor 74 collects and analyzes a several second segment of the patient's cardiac rhythm. The cardiac rhythm analysis program executed by processor 74 is stored in program memory 76. Algorithms of the type implemented by the rhythm analysis program are generally known and disclosed, for example, in the W. A. Tacker Jr. book Defibrillation of the Heart, 1994. If the processor 74 determines that the patient has a nonshockable cardiac rhythm that is not susceptible to treatment by defibrillation pulses (e.g., no pulse rather than a fibrillating rhythm), it initiates the generation of a “Check pulse. If no pulse, give CPR.” voice prompt. One minute after this voice prompt, processor 74 repeats the initiation of the “Do not touch patient. Analyzing rhythm” voice prompt and 30 the associated cardiac rhythm analysis.

When a shockable cardiac rhythm is detected, processor 74 begins a first charge sequence by initiating the generation of a “Charging” voice prompt, and causes high voltage energy delivery system 19 to operate in the charge mode. When the high voltage energy delivery system 19 is charged, processor 74 begins a first shock sequence by initiating the generation of a “Stand clear. Push flashing button to rescue” voice prompt, and the flashing illumination of rescue switch light 19. The operator actuation of rescue switch 40 will then cause processor 74 to operate high voltage energy delivery system 19 in the discharge mode, and results in the application of a defibrillation pulse to the patient to complete the first series of analyze/charge/shock sequences.

Following the first series of analyze/charge/shock sequences, processor 74 times out for a short pause of about five seconds to allow the heart to reestablish its cardiac rhythm before beginning a second series of analyze/charge/shock sequences. The second series of analyze/charge/shock sequences is identical to the first series described above, except the energy content of the defibrillation pulse may be equal to or greater than the energy content of the first pulse. If the second series of analyze/charge/shock sequences ends with the delivery of a defibrillation pulse, processor 74 again times out for a short pause of about five second before beginning a third analyze/charge/shock sequence. The third series is also identical to the first series, but processor 74 controls the high voltage energy delivery system 19 in such a manner as to cause the defibrillation pulse delivered upon the actuation of the rescue switch 40 to have an even higher energy content.

Following the delivery of a defibrillation pulse at the end of the third series of analyze/charge/shock sequences, or after identifying a nonshockable cardiac rhythm, processor 74 initiates the generation of a “Check Pulse. If no pulse, give CPR” voice prompt. Processor 74 then times a one minute CPR period to complete a first set of three series of analyze/charge/shock sequences. Rescue mode operation then continues with additional sets of three series of analyze/charge/shock sequences of the type described above. Processor 74 ends the rescue mode operation of defibrillator 70 when a total of nine series of analyze/charge/shock sequences have been performed, or the defibrillator cover is closed.

Throughout the analyze, charge and shock sequences, processor 74 monitors the impedance present across connector 58 to determine whether electrodes 50 remain properly positioned on the patient. If the monitored impedance is out of range (e.g., too high if the electrodes have come off the patient, or too low if shorted), processor 74 initiates the generation of a “Check Electrodes” voice prompt, and causes high voltage generation circuit 86 to discharge any charge that may be present through internal load 98. Rescue mode operation will resume when processor 74 determines that the electrodes have been properly repositioned on the patient.

Data representative of the operation of the defibrillator and the monitored cardiac rhythm of the patient are stored in event memory 78 during rescue mode operation. Stored data representative of the operation of the defibrillator may include the real time of the occurrence of some or all of the following events: 1) the placement of electrodes on the patient, 2) the initiation of the cardiac rhythm analysis voice prompt, 3) the initiation of the charging voice prompt, 4) the completion of the charge mode operation of,high voltage energy delivery system 19, and 5) the actuation of rescue switch 40. The actual time base of the patient's cardiac rhythm is also stored in memory 78. Following a rescue, the stored data can be retrieved from event memory 78 through the use of a personal computer (PC) (not shown) interfaced to communications port 32.

Although various embodiments are specifically illustrated and described herein, it will be appreciated that modifications and variations of the present invention are covered by the above teachings and are within the purview of the appended claims without departing from the spirit and intended scope of the invention. For example, the defibrillator may comprise the above-described speaker element which operates concurrently with the wireless transmitter 85 and the headphone 56. Such an arrangement offers redundancy in the aural user interface. Also, additional prompting steps or other variations in the aforementioned protocols are within the scope of the present invention.

1. A method for providing instructions to a user for operating an external defibrillator device having a set of electrodes couplable to a patient, said method comprising the steps of:
transmitting over a wireless protocol a voice prompt instructing the user to attach the set of electrodes to the patient; and
transmitting over the wireless protocol at least one additional voice prompt instructing the user to administer defibrillator therapy.

2. The method of claim 1 further comprising the step of transmitting over the wireless protocol a voice prompt instructing the user to administer CPR therapy.

3. The method of claim 2 further comprising the step of transmitting over the wireless protocol a voice prompt instructing the user that a patient assessment sequence is to begin.

4. The method of claim 1 wherein said external defibrillator is a fully automatic external defibrillator.

5. The method of claim 1 wherein said external defibrillator is a semi-automatic external defibrillator.

6. The method of claim 1 wherein said wireless protocol is selected from the group consisting of Bluetooth, IEEE 802.11, IEEE 802.15, IEEE802.16, Near Field Communication—Interface and Protocol (“NFCIP-1”), and HomeRF.

7. The method of claim 1, further comprising the step of transmitting voice prompts to a receiver embedded in a portable device.

8. The method of claim 7 wherein said portable device is selected from the group consisting of a headphone, wireless telephone and a PDA.

9. An electrotherapy device comprising:
a controller;
an energy source;
at least one electrode for providing electrotherapy to a patient;
an energy delivery system operable by the controller to deliver an electrical shock from the energy source to the at least one electrode;
a voice circuit for generating audio prompts initiated by the controller;
a wireless transmitter coupled to the voice circuit for transmitting the audio prompts over a wireless communication protocol.

10. The electrotherapy device of claim 9 further comprising a portable device having a wireless receiver embedded therein, said wireless receiver operating in accordance with the wireless communication protocol over which the wireless transmitter operates.

11. The electrotherapy device of claim 10 wherein said portable device is selected from the group consisting of a headphone, wireless telephone and a PDA.

12. The electrotherapy device of claim 9 wherein said electrotherapy device is an external defibrillator.

13. The electrotherapy device of claim 12 wherein said external defibrillator is a fully automatic external defibrillator.

14. The electrotherapy device of claim 12 wherein said external defibrillator is a semi-automatic external defibrillator.

15. The electrotherapy device of claim 9 wherein said wireless communication protocol employed by the wireless receiver is selected from the group consisting of Bluetooth, IEEE 802.11, IEEE 802.15, IEEE802.16, Near Field Communication—Interface and Protocol (“NFCIP-1”), and HomeRF.
